(19) United States
(12) Patent Application Publication
(10) Pub. No.: US 2018/0093675 A1
(43) Pub. Date: Apr. 5, 2018

(54) Wake Alarm For Vehicles With An Autonomous Mode

(71) Applicant: Ford Global Technologies, LLC, Dearborn, MI (US)

(72) Inventors: Patrick Kevin Holub, Novi, MI (US); Nicholas Patrick Holub, Novi, MI (US)

(21) Appl. No.: 15/282,881

(22) Filed: Sep. 30, 2016

(51) Int. Cl. B60W 50/08; B60W 10/20; B60W 10/04; B60W 50/14; G05D 1/00; B60W 30/14; B60W 30/182; A61B 5/024; A61B 5/18; A61B 5/00

(52) U.S. Cl. CPC B60W 50/082; B60W 10/20; B60W 10/04; B60W 50/14; G05D 1/0061; G05D 1/0088; B60W 2550/20; B60W 30/182; A61B 5/02405; A61B 5/18; A61B 5/6893; B60W 2420/42; B60W 2540/26; B60W 30/14

(57) ABSTRACT

Method and apparatus are disclosed for a wake alarm for vehicles with an autonomous mode. An example disclosed vehicle includes a sensor and a camera to monitor a state of a driver, and a transition manager. The example transition manager, at a first transition location, provides notification to the driver and restores vehicle interior settings from autonomous mode preferences to manual mode preferences. Additionally, the example transition manager, at a second transition location, when the state of the driver indicates that the driver is alert, transfers control of the vehicle to the driver.

### TECHNICAL FIELD

The present disclosure generally relates to semi-autonomous vehicles and, more specifically, a wake alarm for vehicles with an autonomous mode.

### BACKGROUND

Increasingly, vehicles are being equipped with autonomous modes that facilitate navigating a mapped region with sufficient detail or a well marked road or lane of a road. However, a human driver must intervene when the vehicle enters an area that with not sufficiently mapped. For example, large, densely populated regions may be sufficiently mapped while smaller communities may not be.

### SUMMARY

The appended claims define this application. The present disclosure summarizes aspects of the embodiments and should not be used to limit the claims. Other implementations are contemplated in accordance with the techniques described herein, as will be apparent to one having ordinary skill in the art upon examination of the following drawings and detailed description, and these implementations are intended to be within the scope of this application.

Example embodiments are disclosed for a wake alarm for vehicles with an autonomous mode. An example disclosed vehicle includes a sensor and a camera to monitor a state of a driver, and a transition manager. The example transition manager, at a first transition location, provides a notification to a driver and restores vehicle interior settings from autonomous mode preferences to manual mode preferences. Additionally, the example transition manager, at a second transition location, when the state of the driver indicates that the driver is alert, transfers control of the vehicle to the driver.

An example method includes monitoring a state of a driver with a sensor and a camera integrated into a vehicle. The example method includes, at a first transition location providing a notification to the driver, and restoring vehicle interior settings from autonomous mode preferences to manual mode preferences. Additionally, the example method includes, at a second transition location, when the state of the driver indicates that the driver is alert, transfer control of the vehicle to the driver.

An example tangible computer readable medium comprising instruction that, when executed, cause a vehicle to monitor a state of a driver with a sensor and a camera located inside the vehicle. Additionally, the example instruction cause the vehicle to, at a first transition location provide a notification to the driver, and restore vehicle interior settings from autonomous mode preferences to manual mode preferences. The example instructions also cause the vehicle to, at a second transition location, when the state of the driver indicates that the driver is alert, transfer control of the vehicle to the driver.

### BRIEF DESCRIPTION OF THE DRAWINGS

For a better understanding of the invention, reference may be made to embodiments shown in the following drawings. The components in the drawings are not necessarily to scale and related elements may be omitted, or in some instances proportions may have been exaggerated, so as to emphasize and clearly illustrate the novel features described herein. In addition, system components can be variously arranged, as known in the art. Further, in the drawings, like reference numerals designate corresponding parts throughout the several views.

FIGS. 1A and 1B illustrate an interior of a vehicle operating in accordance with the teachings of this disclosure.

FIG. 2 illustrates electronic components of the vehicle of FIGS. 1A and 1B.

FIG. 3 is a flowchart of a method to transition the vehicle of FIGS. 1A and 1B to a manual mode that may be implemented by the electronic components of FIG. 2.

### DETAILED DESCRIPTION OF EXAMPLE EMBODIMENTS

While the invention may be embodied in various forms, there are shown in the drawings, and will hereinafter be described, some exemplary and non-limiting embodiments, with the understanding that the present disclosure is to be considered an exemplification of the invention and is not intended to limit the invention to the specific embodiments illustrated.

Semi-autonomous vehicles are vehicles in which some of the motive functions of the vehicle are handled autonomously by the vehicle. These motive functions include from assisted parking to full navigation without direct driver input (e.g., beyond a destination). Autonomous navigation in urban areas often uses detailed maps of traffic and road features (e.g., lane pattern, traffic signals and signs, turn angles, traffic patterns, etc.). Additionally, autonomous navigation may use markings and signs on well marked roads. Well mapped areas tend to cluster around densely populated urban areas and well marked roads tend to include major intrastate highways and interstate highways. In areas that the detailed map is not available and/or the roads are not well marked, human driver intervention is necessary. For example, a route may originate and terminate in areas that are not sufficiently mapped, but are connected by a well marked interstate highway. In such an example, driver intervention may be used to navigate onto the interstate highway and to navigate the final portion of the route between the interstate highway and the destination. On long road trips, the focus of the driver may drift from the road. Additionally, the vehicle may have one or more features to facilitate the driver doing other activities while the vehicle is in the autonomous mode.

As disclosed below, the vehicle includes features and/or vehicle interior preference settings that are available while the vehicle is in an autonomous mode and change and/or are not available when the vehicle in a non-autonomous mode (sometime referred to as a “manual” mode). For example, when the autonomous portion of a route is long, the features and/or the vehicle interior preference settings may provide conditions (e.g., darkening the tint of windows, positioning the driver's seat back, recessing the steering wheels and/or the pedals, etc.) in which the driver may sleep. On a route, the vehicle determines a first transition point at which the vehicle is to begin transitioning from the autonomous mode to the manual mode. The first transition point is a location on the route that the vehicle is to transition the vehicle features and the vehicle subsystem preference settings from the autonomous mode to the manual mode with so that the driver is in the driving seat and cognizant of the road, the route, and the area surrounding the vehicle before the driver is to take control of the vehicle at a second transition point. In some examples, the first transition point is determined via a navigation program. Additionally or alternatively, an infrastructure node of a vehicle-to-infrastructure (V2I) network broadcasts a message to inform the vehicle of the locations of the first and second transition points. The vehicle includes sensors (e.g., weight sensors, biometric sensors, etc.) and cameras to track the position and state of consciousness (sometimes referred to herein as the “condition”) of the driver.

At the second transition point, if the vehicle determines that the driver is capable of assuming control of the vehicle based on the condition of the driver, the vehicle transfers control of the vehicle to the driver. If, however, the vehicle determines that the driver is not capable of assuming control of the vehicle based on the condition of the driver, the vehicle performs an emergency contingency. The emergency contingency may include pulling the vehicle over to the shoulder of the road and/or into an emergency portion of the road designated for such contingencies. Additionally, in some examples, the vehicle performs mitigating techniques (e.g., activating/increasing the volume of the sound system, increasing the air conditioning blower speed, decreasing the air conditioner temperature setting, etc.) and/or provides instructions for the driver to perform in order to receive control of the vehicle (e.g., placing hands on the steering wheel, directing gaze at the road, etc.).

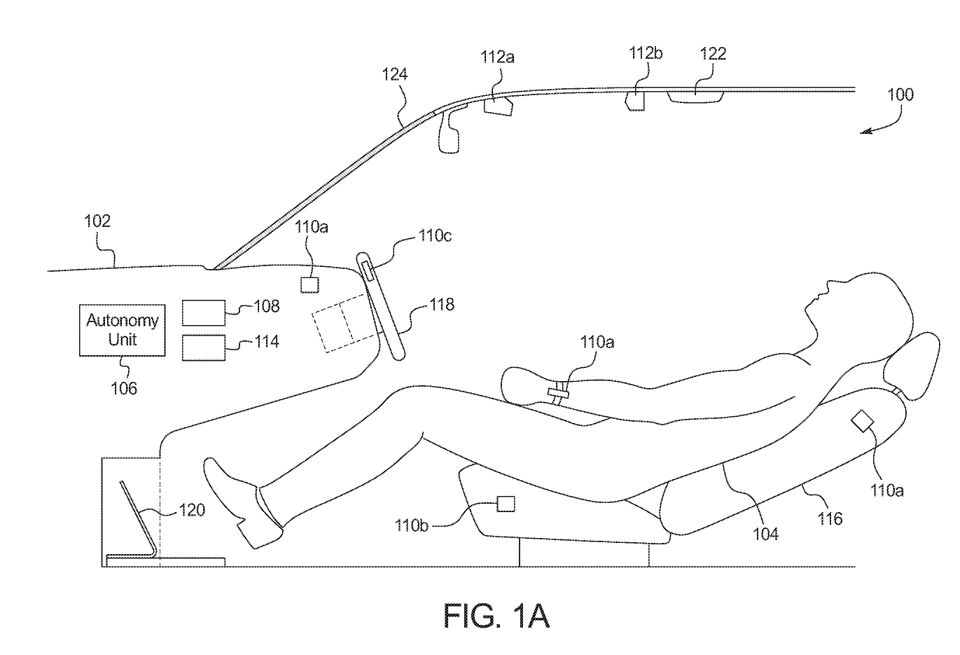

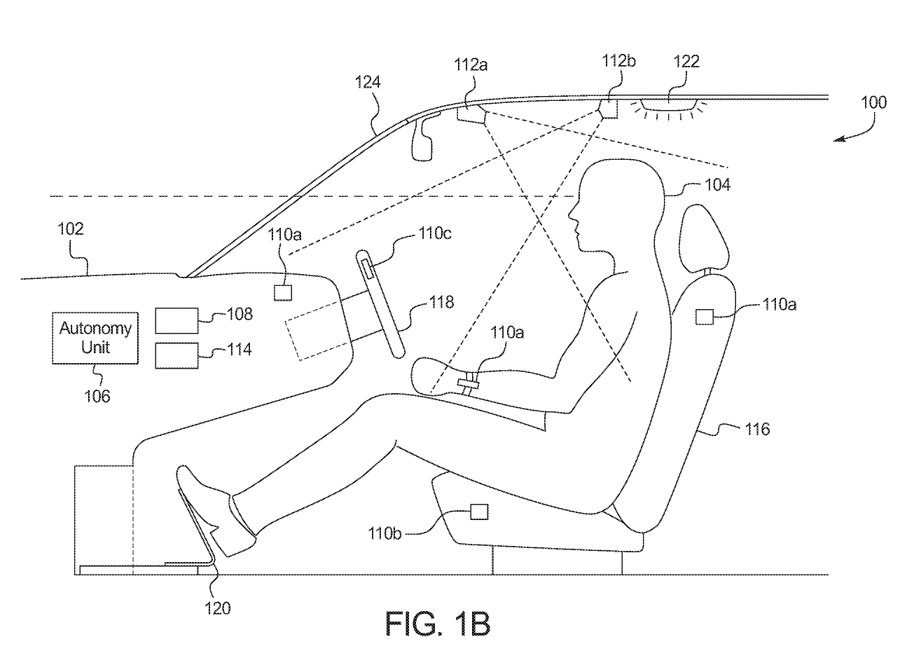

FIGS. 1A and 1B illustrate a cabin 100 of a vehicle 102 (e.g., a car, a truck, a semi-trailer truck, a recreational vehicle, etc.) operating in accordance with the teachings of this disclosure. FIG. 1A illustrates an example of features and/or vehicle interior preference settings in an autonomous mode (sometimes referred to as “autonomous mode preferences”. FIG. 1B illustrates an example of the features and/or the vehicle subsystem preference settings in a manual mode (sometimes referred to as “manual mode preferences”). The vehicle 102 may be a standard gasoline powered vehicle, a hybrid vehicle, an electric vehicle, a fuel cell vehicle, and/or any other mobility implement type of vehicle. The vehicle 102 includes parts related to mobility, such as a powertrain with an engine and/or motors, a transmission, a suspension, a driveshaft, and/or wheels, etc. The vehicle 102 includes an autonomous mode and a manual mode. In the autonomous mode, the vehicle 102 controls the motive functions of the vehicle without intervention from a driver 104 except the input of a destination and other travel preferences. In the manual mode, the driver 104 controls at least some of the motive functions of the vehicle 102. In the illustrated example the vehicle 102 includes an autonomy unit 106, a vehicle-to-everything (V2X) module 108, sensors 110a-110c, cameras 112a and 112b, and a transition manager 114. Additionally, the vehicle 102 includes features and/or vehicle interior preferences that are different in the autonomous mode and the manual mode. In the illustrated examples of FIGS. 1A and 1B, the features and/or the vehicle interior preferences include an angle and/or position of seat 116, angle and/or position of a steering wheel 118, a position of pedals 120, brightness of interior lights 122, and a tint of windows 124 of the vehicle 102. Additionally, in some examples, the features and/or the vehicle interior preferences may include a position of screens, position of a center console display, a position of a footrest, operation of an in-vehicle entertainment system, and/or a position of a shift lever, etc.

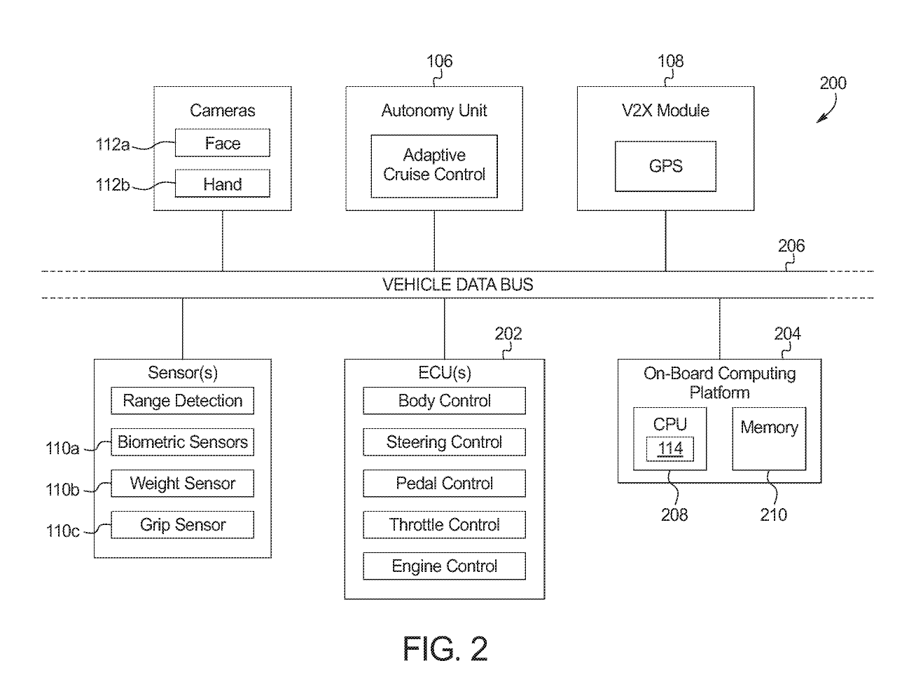

When the vehicle 102 is in the autonomous mode, the autonomy unit 106 controls the motive functions of the vehicle 102 by issuing commands to various electronic control units (ECUs) (e.g., the ECUs 202 of FIG. 2 below). The autonomy unit 106 is coupled to range detection sensors (e.g., ultrasonic sensors, RADAR, LiDAR, infrared sensors, cameras, etc.) to detect characteristics (identity, size, and/or location, etc.) of objects around the vehicle 102 and detect road characteristics (e.g., location and size of lanes, speed limits, etc.). Additionally, the autonomy unit 106 uses navigation data (e.g., lanes, road curvature, road grade, road surface material, speed limits, etc.) about the route. Additionally, in some examples, the autonomy unit 106 coordinates travel (e.g., speeds, gaps between vehicles, etc.) with other vehicles and/or communicates with traffic infrastructure via the V2X module 108. For example, the autonomy unit may include cooperative adaptive cruise control. The autonomy unit 106 may also include other functions to assist the driver 104 to perform routine motive functions when the vehicle 102 is in manual mode, such as assisted parking, adaptive cruise control, lane drift detection, and blind spot detection.

The V2X module 108 includes radio(s) and software to broadcast messages and to establish connections between the vehicle 102, other vehicles (sometimes referred to as vehicle-to-vehicle (V2V) or car-to-car (C2C) communication), infrastructure-based modules (not shown) (sometimes referred to as vehicle-to-infrastructure (V2I) or car-to-infrastructure (C2I) communication), and mobile device-based modules (not shown) (sometimes referred to as vehicle-to-pedestrian (V2P) or car-to-pedestrian (C2P) communication). The V2X module 106 includes a global positioning system (GPS) receiver and an inertial navigation system (INS) to determine and share the location of the vehicle 102 and to synchronize the V2X module 106 with modules of other vehicles and/or infrastructure nodes. An example implementation of a V2X network is the Dedication Short Range Communication (DSRC) protocol. More information on the DSRC network and how the network may communicate with vehicle hardware and software is available in the U.S. Department of Transportation's Core June 2011 System Requirements Specification (SyRS) report (available at http://www.its.dot.gov/meetings/pdf/CoreSystem_SE_SyRS_RevA%20(2011-06-13).pdf), which is hereby incorporated by reference in its entirety along with all of the documents referenced on pages 11 to 14 of the SyRS report. V2X systems may be installed on vehicles and along roadsides on infrastructure. V2X systems incorporating infrastructure information is known as a “roadside” system. V2X may be combined with other technologies, such as Global Position System (GPS), Visual Light Communications (VLC), Cellular Communications, and short range radar, facilitating the vehicles communicating their position, speed, heading, relative position to other objects and to exchange information with other vehicles or external computer systems.

Currently, in the United States, the V2X network is identified under the DSRC abbreviation or name. However, other names are sometimes used, usually related to a Connected Vehicle program or the like. Most of these systems are either pure DSRC or a variation of the IEEE 802.11 wireless standard. However, besides the pure DSRC system it is also meant to cover dedicated wireless communication systems between cars and roadside infrastructure system, which are integrated with GPS and are based on an IEEE 802.11 protocol for wireless local area networks (such as, 802.11p, etc.).

Various sensors may be arranged in and around the vehicle 102 in any suitable fashion. These sensors may, for example, measure properties around the exterior of the vehicle 102. Additionally, some of these sensors may be mounted inside the cabin of the vehicle 102 or in the body of the vehicle 102 (such as, the engine compartment, the wheel wells, etc.) to measure properties in the interior of the vehicle 102. For example, such sensors may include accelerometers, odometers, tachometers, pitch and yaw sensors, wheel speed sensors, microphones, and tire pressure sensors, etc. In the illustrated example, the sensors 110a-110c monitor the driver 104 to determine indications of position and state of consciousness (e.g., asleep, drowsy, alert, etc.) of the driver 104. The sensors 110a-110c include biometric sensors 110a, a weight sensor 110b, and a grip sensor 110c. The biometric sensors 110a include sensors that measure physiological properties of the driver 104, such as a heart rate monitor, respiration monitor, and/or a body temperature sensor, etc. For example, the drowsiness of the driver may be determined by the heart rate variability which measures the changes of the intervals from beat to beat. In such an example, the ratio of low frequencies to high frequencies decrease as the driver 104 becomes drowsy. In some examples, the heart rate monitor is a capacitive sensor in the steering wheel 118 and/or the seat 116. In some examples, the biometric sensors 110a include wearable devices (e.g., a smart watch, a fitness tracker, etc.) that are communicatively coupled to the vehicle 102 (e.g., via a Bluetooth® connection, etc.). The weight sensor 110b determines measures whether the driver 104 is in the seat 116. In some examples, the weight sensor 110b outputs a rolling average, over a period of time (e.g., ten seconds, etc.), of the an indication as to whether the driver 104 is in the seat 116 to take into account normal shifting of weight while the driver 104 is driving. The grip sensor 110c determines whether the driver 104 has his/her hands on the steering wheel 118. The grip sensor 110c differentiates between a body part (e.g., an elbow, etc.) on the steering wheel 118 and the hands of the driver 104 being on the steering wheel. Examples of grip sensors 110c in the steering wheel 118 are described in U.S. application Ser. No. 15/158,863, entitled “Driver Detection Steering Wheel,” filed May 19, 2016, which is hereby incorporated by reference herein in its entirety.

The cameras 112a and 112b monitor the driver 104 for indications of the position and the state of consciousness of the driver 104. In the illustrated example, a face camera 112a is positioned to take images of the face of the driver 104. For example, the face camera 112a may be located on a rear view mirror or an overhead center console. The face camera 112a detects (a) the position of the head of the driver 104, (b) the state of the eyes (e.g., open, partially open, or closed) of the driver 104, and/or (c) the direction of the gaze of the driver 104. Indications that the driver 104 is asleep or drowsy include closure or drooping of the eyelids (e.g., as measured by percentage of eyelid closure over a pupil over time), frequency of yawning, a direction of a gaze of the driver 104 that is not on the road, and/or a lowered position and/or quick jerk of the head of the driver 104, etc. Additionally, the face camera 112a detects whether the driver is in the seat 116 (e.g., the position of the driver). For example, because some vehicles 102 (such as recreational vehicles) may be configured to facilitate movements within the vehicle 102 with relative ease, the driver may not remain in the seat 116 while the vehicle 102 is in the autonomous mode. In some examples, the face camera 112a also detects whether the person in the seat 116 is an appropriate size for a driver (e.g., not a child).

The hand camera 112b is positioned to monitor the steering wheel 118 to determine whether the hands of the driver 104 are on the steering wheel 118. The hand camera 112b differentiates between the hands of the driver 104 and other body parts (e.g., the elbow, etc.) that may be placed on the steering wheel 118. The hand camera 112b may be located in any location that provides the hand camera 112b a view of the steering wheel 118 when the driver 104 is in a driving position, such as on the rear view mirror or the overhead center console.

The transition manager 114 transitions the vehicle 102 between the autonomous mode and the manual mode. When the vehicle 102 transitions to the autonomous mode, the transition manager 114 autonomously or at the direction of the driver 104, transitions the subsystems of the vehicle 102 to reflect preferences of the driver 104. For example, the transition manager 114 may increase the tint of the windows 124, dim the interior lights 122 and the dashboard display, recess the pedals 120 into floor, and/or recess the steering wheel 118 into the dashboard, etc. Additionally, the transition manager 114 (a) determines a location of a first transition point and a location of a second transition point, (b) determines when the vehicle 102 is at the first transition point, (c) provides an audio, visual and/or haptic notification to the driver 104 (c) transitions the features and/or vehicle interior to settings for manual driving, (d) determines whether the state of consciousness of the driver 104 indicates that the driver 104 is able to drive the vehicle 102, and (e) when the vehicle 102 reaches a second transition point, reacts based on whether the driver 104 is able to drive the vehicle 102.

The transition manager 114 determines the location of the first transition point and the location of the second transition point. In some examples, the transition manager 114 determines the locations based on a route of the vehicle 102 and first and second transition points defined by navigation data. Additionally or alternatively, the transition manager 114 determines the second transition point based on the location of the first transition point and speed of the vehicle 102. Additionally or alternatively, the infrastructure nodes along the road broadcast messages, via V2I communication, that indicate the location of the first and/or second transition points. For example, construction, an accident, or a natural disaster may cause a temporary transition point that may not timely be reflected in the navigation data. In such an example, the infrastructure nodes may be affixed to infrastructure to provide notice of the transition points.

The transition manager 114 determines when the vehicle 102 is at the first transition point. In some examples, the transition manager 114 determines the location of the vehicle 102 via the GPS receiver of the V2X module 108. Alternatively, in some examples, the vehicle 102 includes a separate GPS receiver. In some examples, the supplements with GPS data with geometry data received from range detection sensors to determine the location of the vehicle 102 in areas (such as urban canyons, etc.) wherein reception of the GPS receiver is poor. When the vehicle 102 is at the location of the first transition point, the transition manager 114 provides an audio, visual and/or haptic notification to the driver 104. In some examples, an intensity of the audio, visual and/or haptic notification is set to wake the driver 104 in case the driver 104 is sleeping. Alternatively or additionally, the intensity of the audio, visual and/or haptic notification is set based on whether the driver 104 is awake or asleep (e.g., as determined by the face camera 112a, etc.). For example, a haptic notification may include vibrating the seat 116.

The transition manager 114 transitions the features and/or vehicle interior preferences between the autonomous mode and the manual mode. In the illustrated example of FIG. 1A, the features and/or vehicle subsystems are set into modes for when the vehicle 102 is in the autonomous mode. Some features and/or vehicle subsystems are adjusted for occupant comfort and some features and/or vehicle subsystems are adjusted to prevent the driver 104 from interfere with the motive functions of the vehicle 102 while the vehicle 102 is in the autonomous mode. In the illustrated example, (i) the seat 116 is reclined, (ii) the steering wheel 118 is recessed into the dashboard, (iii) the pedals 120 are recessed into the floor panel, (iv) the interior lights 122 are dimmed, and (v) the windows 124 are tinted. FIG. 1B illustrates the vehicle 102 transitioned into the manual mode. In the illustrated example of FIG. 1B, (i) the seat 116 is in an upright position, (ii) the steering wheel 118 is in a driving position, (iii) the pedals 120 are in driving positions, (iv) the interior lights 122 (e.g., the dashboard display, the center console display, etc.) are illuminated, and (v) the tint of the windows 124 is reduced. In some examples, the features and/or vehicle subsystem settings are based on preferences (e.g., position and angle of the seat 116, position and angle of the steering wheel 118, positions of the pedals 120, etc.) associated with the driver 104. Additionally, when the transition manager 114 transitions the vehicle into the manual mode, the transition manager 114 activates the sensors 110a-110c and the cameras 112a and 112b.

The transition manager 114 determines whether the state of consciousness of the driver 104 indicates that the driver 104 is able to drive the vehicle 102 based on measurements of the driver 104 by the sensors 110a-110c and the cameras 112a and 112b. In some examples, the transition manager 114 uses the measurements from the biometric sensors 110a to determine whether the driver 104 is sleeping, drowsy, or alert. In some examples, the transition manager 114 uses measurements from more than one sensor 110a-110c and/or camera 112a and 112b in order to determine that the driver is alert (e.g., not sleeping or drowsy) and therefore able to resume control of the vehicle 102. For example, the transition manager 114 based the determination on the grip sensor 110c and the face camera 112a. In some such examples, the transition manager 114 determines that the driver 104 is unable to control the vehicle 102 if any of the sensors 110a-110c and/or the cameras 112a and 112b determines that driver is asleep or drowsy. In some examples, the transition manager 114 may initially determine whether the driver 104 is sitting in the seat 116 based on the weight sensor 110b and/or the face camera 112a.

When the vehicle 102 reaches the second transition point, transition manager 114 reacts based on whether the driver 104 is able to drive the vehicle 102. When the transition manager 114 determines, based on the measurements from the sensors 110a-110c and/or the cameras 112a and 112b, that the driver 104 is (a) sitting in the seat 116, (b) gripping the steering wheel 118, and (c) alert, the transition manager 114 transitions the vehicle 102 so, for example, steering control receives input from the steering wheel 118 and throttle and brake controls receive input from the pedals 120. When the transition manager 114 determines that the driver 104 is either (a) not in the seat 116, (b) not gripping the steering wheel 118, or (c) drowsy or asleep, the transition manager 114 initiates an emergency contingency. The emergency contingency, for example. may include removing the vehicle 102 from the roadway. For example, the transition manager 114 may direct the autonomy unit 106 to navigate the vehicle 102 onto the shoulder of the road, into a rest area, or into a location designated for the vehicle 102 to wait (such as a ride share parking lot, an emergency turn off, etc.). In some examples, the transition manager 114 may also contact assistance (e.g., a vehicle manufacturer concierge service, emergency assistance, an emergency contact, etc.).

FIG. 2 illustrates electronic components 200 of the vehicle 102 of FIGS. 1A and 1B. In the illustrated example, the electronic components 200 include the autonomy unit 106, the V2X module 108, the sensors 110a-110c, the cameras 112a and 112b, electronic control units (ECUs) 202, an on-board computing platform 204, and a vehicle data bus 206.

The ECUs 202 monitor and control the subsystems of the vehicle 102. The ECUs 202 communicate and exchange information via a vehicle data bus (e.g., the vehicle data bus 206). Additionally, the ECUs 202 may communicate properties (such as, status of the ECU 202, sensor readings, control state, error and diagnostic codes, etc.) to and/or receive requests from other ECUs 202. Some vehicles 102 may have seventy or more ECUs 202 located in various locations around the vehicle 102 communicatively coupled by the vehicle data bus 206 and/or dedicated signal wires. The ECUs 202 are discrete sets of electronics that include their own circuit(s) (such as integrated circuits, microprocessors, memory, storage, etc.) and firmware, sensors, actuators, and/or mounting hardware. In FIG. 2, the example ECUs 202 include a body control module, a steering control module, a pedal control module, a throttle control module, and an engine control module. The ECUs 202 control the subsystems that affect the motive functions of the vehicle 102 and control the subsystems associated with the features and/or the vehicle subsystem preferences of the autonomous and manual modes. For example, the body control module may control the tint of the windows and the steering wheel control module may control the position and angle of the steering wheel 118, etc.

The on-board computing platform 204 includes a processor or controller 208 and memory 210. In some examples, the on-board computing platform 204 is structured to include the transition manager 114. Alternatively, in some examples, the transition manager 114 may be incorporated into another ECU 202 with its own processor and memory, such as the autonomy unit 106. The processor or controller 208 may be any suitable processing device or set of processing devices such as, but not limited to: a microprocessor, a microcontroller-based platform, a suitable integrated circuit, one or more field programmable gate arrays (FPGAs), and/or one or more application-specific integrated circuits (ASICs). The memory 210 may be volatile memory (e.g., RAM, which can include non-volatile RAM, magnetic RAM, ferroelectric RAM, and any other suitable forms); non-volatile memory (e.g., disk memory, FLASH memory, EPROMs, EEPROMs, memristor-based non-volatile solid-state memory, etc.), unalterable memory (e.g., EPROMs), read-only memory, and/or high-capacity storage devices (e.g., hard drives, solid state drives, etc). In some examples, the memory 210 includes multiple kinds of memory, particularly volatile memory and non-volatile memory.

The memory 210 is computer readable media on which one or more sets of instructions, such as the software for operating the methods of the present disclosure can be embedded. The instructions may embody one or more of the methods or logic as described herein. In a particular embodiment, the instructions may reside completely, or at least partially, within any one or more of the memory 210, the computer readable medium, and/or within the processor 208 during execution of the instructions.

The terms “non-transitory computer-readable medium” and “computer-readable medium” should be understood to include a single medium or multiple media, such as a centralized or distributed database, and/or associated caches and servers that store one or more sets of instructions. The terms “non-transitory computer-readable medium” and “computer-readable medium” also include any tangible medium that is capable of storing, encoding or carrying a set of instructions for execution by a processor or that cause a system to perform any one or more of the methods or operations disclosed herein. As used herein, the term “computer readable medium” is expressly defined to include any type of computer readable storage device and/or storage disk and to exclude propagating signals.

The vehicle data bus 206 communicatively couples the autonomy unit 106, the V2X module 108, the sensors 110a-110c, the cameras 112a and 112b, ECUs 202, and the on-board computing platform 204. In some examples, the vehicle data bus 206 includes one or more data buses. The vehicle data bus 206 may be implemented in accordance with a controller area network (CAN) bus protocol as defined by International Standards Organization (ISO) 11898-1, a Media Oriented Systems Transport (MOST) bus protocol, a CAN flexible data (CAN-FD) bus protocol (ISO 11898-7) and/a K-line bus protocol (ISO 9141 and ISO 14230-1), and/or an Ethernet™ bus protocol IEEE 802.3 (2002 onwards), etc.

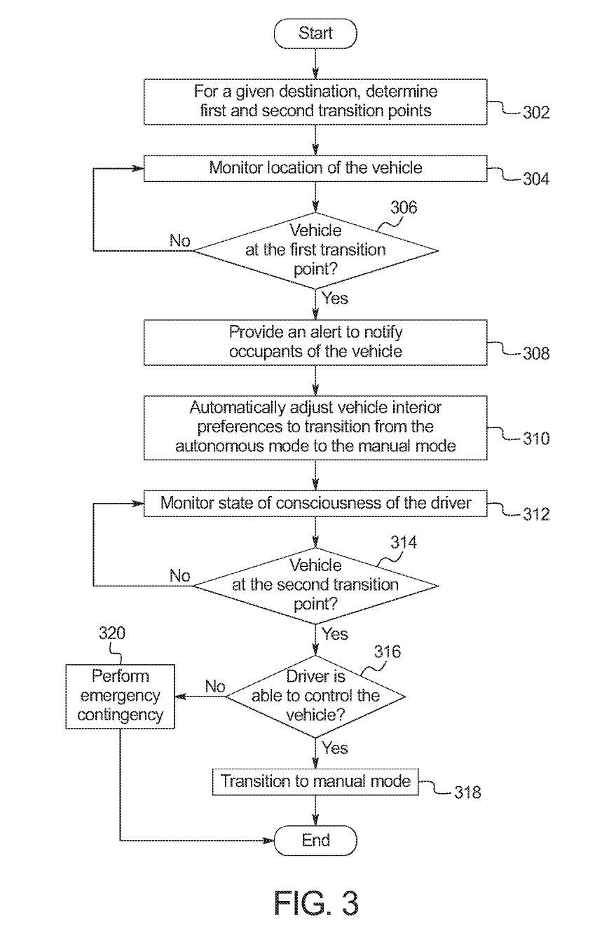

FIG. 3 is a flowchart of a method to transition the vehicle 102 of FIGS. 1A and 1B to a manual mode that may be implemented by the electronic components 200 of FIG. 2. Initially, at block 302, the transition manager 114, for a given destination, determines a first and second transition point at which to (1) transition from an automatic mode to a manual mode and (2) to transfer control of the vehicle 102 to the driver 104. At block 304, the transition manager 114 monitors the location of the vehicle 102. At block 306, the transition manager 114 determines whether the vehicle 102 is at the first transition point. If the vehicle 102 is at the first transition point, the method continues to block 308. Otherwise, if the vehicle 102 is not at the first transition point, the method returns to block 304.

At block 308, the transition manager 114 provides an audio, visual and/or haptic notification to the driver 104 to notify the driver 104 that the vehicle 102 has reached the first transition point. In some examples, the alert is set to wake the driver 104 when measurements from the sensors 110a-110a and/or cameras 112a and 112b indicate that the driver 104 is sleeping. At block 310, the transition manager 114 automatically adjusts the vehicle subsystems to transition from the autonomous mode to the manual mode. For example, the transition manager 114 may reposition the steering wheel 118 and transition the seat 116 from a laid back position to an upright position. At block 312, the transition manager 114 monitors, via the sensors 110a-110c and/or the cameras 112a and 112b, the position (e.g., in the seat 116, etc.) and state of consciousness (e.g., alert, drowsy, sleeping, etc.) of the driver 104. At block 314, the transition manager 114 determines whether the vehicle 102 is at the second transition point. If the vehicle 102 is at the second transition point, the method continues at block 316. Otherwise, if the vehicle 102 is not at the second transition point, the method returns to block 312.

At block 316, the transition manager 114 determines whether the driver 104 is able to control the vehicle 102 based on the measurements from sensors 110a-110b and/or the cameras 112a and 112b. In some examples, the transition manager 114 determines that the driver 104 is able to take control of the vehicle 102 if the driver is determined to be alert. If the driver is able to take control of the vehicle 102, the method continues at block 318. Otherwise, if the driver is not able to take control of the vehicle 102, the method continues at block 320. At block 318, the transition manager 114 transitions the vehicle 102 to manual mode. At block 320, the transition manager 114 performs an emergency contingency.

The flowchart of FIG. 3 is representative of machine readable instructions stored in memory (such as the memory 210 of FIG. 2) that comprise one or more programs that, when executed by a processor (such as the processor 208 of FIG. 2), cause the vehicle 102 to implement the example transition manager 114 of FIGS. 1 and 2. Further, although the example program(s) is/are described with reference to the flowchart illustrated in FIG. 3, many other methods of implementing the example transition manager 114 may alternatively be used. For example, the order of execution of the blocks may be changed, and/or some of the blocks described may be changed, eliminated, or combined.

In this application, the use of the disjunctive is intended to include the conjunctive. The use of definite or indefinite articles is not intended to indicate cardinality. In particular, a reference to “the” object or “a” and “an” object is intended to denote also one of a possible plurality of such objects. Further, the conjunction “or” may be used to convey features that are simultaneously present instead of mutually exclusive alternatives. In other words, the conjunction “or” should be understood to include “and/or”. The terms “includes,” “including,” and “include” are inclusive and have the same scope as “comprises,” “comprising,” and “comprise” respectively.

The above-described embodiments, and particularly any “preferred” embodiments, are possible examples of implementations and merely set forth for a clear understanding of the principles of the invention. Many variations and modifications may be made to the above-described embodiment(s) without substantially departing from the spirit and principles of the techniques described herein. All modifications are intended to be included herein within the scope of this disclosure and protected by the following claims.

What is claimed is:

1. A vehicle comprising:
a sensor and a camera to monitor a state of a driver; and
a transition manager to:
at a first transition location, provide a notification to the driver, and restore vehicle interior settings from autonomous mode preferences to manual mode preferences; and
at a second transition location, when the state of the driver indicates that the driver is alert, transfer control of the vehicle to the driver.

2. The vehicle of claim 1, wherein the sensor includes a grip sensor located in a steering wheel to detect when hands of the driver are gripping the steering wheel, and wherein the hands of the driver gripping the steering wheel being indicative that the state of the driver is alert.

3. The vehicle of claim 1, wherein the sensor includes a heart rate monitor located in a seat to detect changes of intervals from beat to beat of the heart of the driver, wherein a decreasing ratio of low frequency beats to high frequency beats is indicative that the driver is not alert.

4. The vehicle of claim 1, wherein the camera is positioned to capture images of a face of the driver to determine whether the driver is alert based on facial features captured by the camera.

5. The vehicle of claim 1, wherein the camera is positioned to capture images of a steering wheel to determine whether the driver is alert based on a grip of hands of the driver on the steering wheel.

6. The vehicle of claim 1, wherein to determine that the driver is alert, the transition manager is to determine that first measurements from the sensor and second measurements from the camera are indicative of the driver being alert.

7. The vehicle of claim 1, wherein at the second transition location, the transition manager is to, when the state of the driver indicates that the driver is not alert:
instruct an autonomy unit to remain in autonomous mode; and
perform an emergency contingency plan to pilot the vehicle off a roadway.

8. The vehicle of claim 1, including a vehicle-to-everything module and wherein the transition manager is to determine coordinates of the first and second transition points from an infrastructure node proximate the first transition location.

9. A method comprising:
monitoring, with a processor, a state of a driver with a sensor and a camera integrated into a vehicle;
at a first transition location:
providing a notification to the driver, and
restoring vehicle interior settings from autonomous mode preferences to manual mode preferences; and
at a second transition location, when the state of the driver indicates that the driver is alert, transfer control of the vehicle to the driver.

10. The method of claim 9, wherein the sensor includes a grip sensor located in a steering wheel, and wherein monitoring the state of the driver includes detecting when hands of the driver are gripping the steering wheel.

11. The method of claim 9, wherein sensors include a heart rate monitor located in a seat, wherein monitoring the state of the driver includes detecting changes of intervals from beat to beat of a heart of the driver, wherein a decreasing ratio of low frequency beats to high frequency beats is indicative that the driver is not alert.

12. The method of claim 9, wherein the camera is positioned to capture images of a face of the driver, and wherein monitoring the state of the driver includes determining whether the driver is alert based on facial features captured by the camera.

13. The method of claim 9, wherein the camera is positioned to capture images of a steering wheel, and wherein monitoring the state of the driver includes determining whether the driver is alert based on a grip of hands of the driver on the steering wheel.

14. The method of claim 9, including determining that the driver is alert based on first measurements from the sensor and second measurements from the camera, are indicative of the driver being alert.

15. The method of claim 9, including, at the second transition location, when the state of the driver indicates that the driver is not alert:
instructing an autonomy unit to remain in autonomous mode; and
performing an emergency contingency plan to pilot the vehicle off a roadway.

16. The vehicle of claim 9, including determining coordinates of the first and second transition points, via a vehicle-to-everything module, from an infrastructure node proximate the first transition location.

17. A tangible computer readable medium comprising instruction that, when executed, cause a vehicle to:
monitor a state of a driver with a sensor and a camera located inside the vehicle;
at a first transition location:
provide an alert to a driver, and
restore vehicle interior settings from autonomous mode preferences to manual mode preferences; and
at a second transition location, when the state of the driver indicates that the driver is alert, transfer control of the vehicle to the driver.
